(19) United States
(12) Patent Application Publication
(10) Pub. No.: US 2011/0165578 A1
(43) Pub. Date: Jul. 7, 2011

(54) METHODS FOR DETECTING AND QUANTIFYING OVERSULFATED GLYCOSAMINOGLYCANS

(76) Inventors: Daniela Verthelyi, Potomac, MD (US); Maria Cecilia Tami, Rockville, MD (US); Montserrat Puig, Bethesda, MD (US)

(21) Appl. No.: 13/062,919

(22) PCT Filed: Sep. 8, 2009
(86) PCT No.: PCT/US2009/056263

§ 371 Date: Mar. 8, 2011

Related U.S. Application Data
(60) Provisional application No. 61/095,562, filed on Sep. 9, 2008.

(51) Int. Cl. C12Q 1/68; C12Q 1/48

(52) U.S. Cl. 435/6.12; 435/15

(57) ABSTRACT

The invention relates to novel methods for detecting and/or quantifying oversulfated or persulfated glycosaminoglycans based on inhibition of nucleic acid polymerases. The methods can be utilized to detect and quantify oversulfated or persulfated glycosaminoglycans in pharmaceutical preparations, such as heparin preparations or therapeutic medical devices. When used to detect or quantify oversulfated glycosaminoglycans in heparin containing solutions, the samples are prepared by treatment with heparinases to degrade the heparin. Titration of the inhibition of the activity of the polymerases allows quantitation of the oversulfated glycosaminoglycans in the sample.

### CROSS REFERENCE TO RELATED APPLICATION(S)

Benefit is claimed to U.S. Provisional Application 61/095,562, filed Sep. 9, 2008, which is incorporated herein by reference in its entirety.

### FIELD

This disclosure relates to methods for detecting and/or quantifying oversulfated glycosaminoglycans, based on inhibition of nucleic acid polymerases and resistance to enzymatic degradation. Further, it relates to use of these methods to screen and quantify pharmaceutical preparations, such as heparin preparations, for oversulfated contaminants.

### BACKGROUND

Heparin is a naturally occurring acidic carbohydrate produced commercially from extracts of animal tissues, such as bovine lung or porcine intestine. Heparin and low molecular heparins are used in the treatment of a wide range of diseases in addition to their classic anticoagulant activity (Dudas et al., Neurodegen. Dis. 5: 200-205, 2008; Sjalander et al., J. of Internal Med. 263: 52-60, 2008; Falanga & Marchetti, Semin. Thromb. Hemost., 688-694, 2007). They are also used to coat many medical devices, such as catheters, syringes, stents, and filters. Indeed, millions of doses of heparin are dispensed every month. The most significant adverse events linked to heparin have traditionally been increased bleeding and heparin-induced thrombocytopenia (Baglin, J. Clin. Pathol., 54: 272-274, 2001).

Recently, certain lots of heparin have been associated with serious side effects indicative of an allergic-type reaction. Between Jan. 1, 2007 and Apr. 13, 2008, the United States Food and Drug Administration (FDA) received over 700 reports of adverse events in patients receiving heparin as part of their dialysis treatment or surgical procedures. Adverse events included severe hypotension, vasodilation, facial swelling, tachycardia, urticaria, nausea, vomiting, diarrhea, and abdominal pain, and resulted in over 80 deaths. Researchers at the Centers for Disease Control determined that the adverse events were associated with the receipt of heparin sodium for injection (1000 U/ml, in 10 ml and 30 ml multidose vials), manufactured by Baxter Healthcare. As a result, Baxter Healthcare issued recalls for all remaining lots and doses of its multidose and single-dose vials of heparin sodium for injection and HEP-LOCK® heparin flush products. This was followed by recalls for a number of medical devices that contain or are coated with heparin. In March 2008, a similar recall was issued by Rotexmedica GmbH Arzneimittelwerk in Trittau, Germany and since then, suspect lots have been identified in 11 other countries. This indicated an extensive problem with heparin manufacture that was unlikely to be restricted to a single source.

Using multidimensional nuclear magnetic resonance (H-NMR), enzymatic digestion followed by high-performance liquid chromatography, and liquid chromatography with mass spectrometry, Guerrini and colleagues identified an unusual oversulfated form of chondroitin sulfate (OS-CS) as a contaminant present in suspect lots of heparin (Guerrini et al., Nat. Biotech., 26: 669-675, 2008). The OS-CS contained a tetrasulfated disaccharide unit consisting of glucuronic acid linked to N-acetyl-D-galactosamine that was not evident in lots of heparin that were not linked to adverse events (Guerrini et al., Nat. Biotech., 26: 669-675, 2008). Kishimoto and colleagues (Kishimoto et al., N. Engl. J. Med., 358:2457-2467, 2008) subsequently were able to partially reproduce the clinical syndrome in a porcine model by inoculating a large dose of the pure contaminant (5 mg/kg i.v. in bolus) suggesting that the presence of OS-CS was linked to or possibly responsible for the adverse events.

Proton nuclear magnetic resonance (H-NMR spectroscopy) and capillary electrophoresis (CE) were identified by the FDA as tests available to assess the presence of OS-CS contaminant in products containing heparin sodium (on-line at fda.gov/cdrh/safety/heparin-notice.pdf 2008). However, OS-CS is only one of numerous oversulfated compounds of animal, vegetable, insect, or completely synthetic origin that could potentially be designed to co-purify and co-elute with heparin (Kishimoto et al., N. Engl. J. Med., 358:2457-2467, 2008; Lindahl et al., J. Med. Chem. 48: 349-352, 2005; Maruyama et al., Carb. Res., 306: 35-431998; Chen et al., J. Biol. Chem., 280: 42817-42825, 2005; Linhardt et al., Semin. Thromb. Hemost. 453-465, 2007). Most of these synthetic compounds have anti-coagulant activity by current US pharmacopeia (clotting-based) tests and could be designed to give similar spectra by H-NMR as heparin, avoiding identification by the tests currently in place. Such compounds would require methods such as high field spectra NMR for identification (Chen et al., J. Biol. Chem., 280: 42817-42825, 2005). Additionally, for finished dosage forms, traditional tests such as H-NMR or CE cannot determine the presence of contaminant without lyophilizing and concentrating each sample, and may not be suitable for testing finished medical devises. There is therefore a demonstrated need to develop other assay methods for detecting oversulfated compounds.

### SUMMARY

Provided herein is a simple yet highly sensitive in vitro method to detect oversulfated GAGs in a preparation based on inhibition of nucleic acid polymerase activity and resistance to enzymatic degradation. This method can be used to screen heparin lots not only for OS-CS, the contaminant associated with recent adverse events, but for other oversulfated glycosaminoglycans (GAGs). Also provided are methods for quantifying oversulfated GAGs. The described methods can also be used for detecting highly sulfated compounds (such as oversulfated GAGs) in preparations without heparin

Because the amount of starting material required for the described polymerase inhibition assays is low, the disclosed method provides an advantage over CE and H-NMR, by enabling contamination testing of low concentrated heparin samples and heparin-coated devices, without extensive sample pooling and concentration.

In one embodiment, there is provided an in vitro method of detecting (and optionally quantifying) oversulfated glycosaminoglycan, for instance in a medical preparation such as a preparation containing heparin.

Another method for detecting oversulfated glycosaminoglycans involves treating a sample from a preparation (e.g., a heparin lot or other medical preparation) with sufficient heparinase to substantially degrade any heparin therein, thereby producing a heparinase-treated sample, which is then used to assay the activity of a nucleic acid polymerase. Polymerase activity in the heparinase-treated sample is then compared to the activity of the same polymerase in the absence of the heparinase-treated sample, wherein a measurable reduction of the nucleic acid polymerase activity in the presence of heparinase-treated sample indicates that oversulfated glycosaminoglycan is present in the preparation.

Also provided are methods for determining the relative quantity of an oversulfated glycosaminoglycan that is present in a preparation, particularly a preparation containing heparin. In such methods, the preparation is treated with heparinase then used to assay for nucleic acid polymerase activity, and the polymerase activity is compared to the activity of the same nucleic acid polymerase in the presence of known quantities of oversulfated glycosaminoglycan, wherein a similar reduction in the nucleic acid polymerase activity in the heparinase-treated sample and the nucleic acid polymerase activity in one of the known quantities of oversulfated glycosaminoglycan indicates the relative quantity of glycosaminoglycan in the preparation.

Optionally, the methods described herein can be carried out in high throughput format.

Also provided are kits for carrying out the described methods of detecting or quantifying oversulfated GAG. Examples of such kits include a container containing heparinase, nucleic acids (usually in a separate container), a nucleic acid polymerase (usually in a separate container), and instructions for comparing results of the screening method with a standard to provide a conclusion about the presence or quantity of oversulfated glycosaminoglycan contamination in a preparation.

The foregoing and other features and advantages will become more apparent from the following detailed description of several embodiments, which proceeds with reference to the accompanying figures.

### BRIEF DESCRIPTION OF THE FIGURES

FIG. 1 shows the effect of glycosaminoglycans on amplification of a representative 18S DNA template. The effect of heparin on Taq polymerase activity was evaluated by quantitative real time PCR. Heparin lots were treated with heparinase I (1:1 U/U) for two hours at 25° C. or left untreated. Gene amplification was evaluated by real time PCR using the Taqman® Gene Expression assay for human 18S rRNA (20×) and 2× Universal PCR master mix (ABI) in a 25 μl reaction in the presence of increasing concentrations (0.05 to 100 mU) of heparin (heparinase-treated or untreated). FIG. 1A is a chart showing the effects of heparin and heparinase-treated heparin on 18S amplification. FIG. 1B is a chart comparing 3 final drug product lots of heparin characterized as contaminated by H-NMR and CE (B1-B3) with 3 control heparin lots (C1-C3). FIG. 1C is a chart showing the effect of synthetic OS-CS on 18S amplification in the presence and absence of heparinase-treated heparin. FIG. 1D shows the effect of natural and oversulfated synthetic glycosaminoglycans on 18S amplification by PCR. Key: DS: dermatan sulfate, HS: Heparan sulfate, CS-A & E: Chondroitin sulfate type A & E respectively, OS-HS: oversulfated heparan sulfate, OS-DS: oversulfated dermatan sulfate; OS-CS: oversulfated chondroitin sulfate; N/T: not tested.

FIG. 2 shows the sensitivity of the Taq polymerase assay. FIG. 2A is a chart showing that active pharmaceutical ingredient (API) or final drug product (FDP) similarly inhibit Taq polymerase activity. Three lots of API and their corresponding FDP were screened by Taq polymerase inhibition. API was diluted in RNAse free water. As shown, similar degrees of amplification were observed indicating that either API or FDP can be used as the starting material for screening heparin. FIG. 2B is a chart showing that addition of OS-CS (rows B &C; 30 ng) or heparin (rows D &F; 25 mU of uncontaminated lot C1) to cDNA or to the RNA used to generate the cDNA resulted in similar Taq inhibition. Treatment of heparin but not OS-CS with heparinase for 2 hours at room temperature restores gene amplification (polymerase activity) (rows E & G). Also shown is cDNA treated with heparinase buffer (row A).

FIG. 3 shows the screening of heparin samples for oversulfated contaminants. FIG. 3A shows that eight blinded lots of heparin (Bl #1-8) were treated with heparinase and tested for gene amplification by Taqman® PCR at 100 mU, 25 mU or 6.25 mU of heparin. FIG. 3B is a comparison of CE (left spectra) and N acetyl regions of H-NMR (right spectra) profiles for selected samples (Blind #5, #8, and #2 are shown). For the N-acetyl region of H-NMR, the heparin signals at 2.04 ppm, dermatan sulfate at 2.08 ppm, and OS-CS at 2.16 ppm are as previously described (Guerrini et al., Nat. Biotech., 26: 669-675, 2008). For sample #5, CE shows a sharp peak for the contaminant before heparin (2.08 ppm) corresponding to DS. For sample #8, a contaminant is evident by CE as a sharp peak after the heparin and confirmed by H-NMR with a proton peak at 2.16 ppm (arrow) in the N-acetyl region. For sample #2, CE shows no contaminant while H-NMR shows a small peak consistent with the acetyl proton shift of OS-CS. High field (500 MHz) H-NMR is more sensitive than CE for low contaminant levels. FIG. 3C is a plot correlating estimated OS-CS content as determined by PCR inhibition and by capillary electrophoresis. The % OS-CS (w/w) was estimated based on the assumption that 500 pg of OS-CS completely block Tag polymerase (C_{T} value of >35) and 1 mU of heparin has 6.25 ng of active pharmaceutical ingredient.

FIG. 4 is the N-acetyl region of proton NMR spectra for dermatan sulfate (DS) and heparin sulfate (HS) purified from mucosa, and the oversulfated synthetic corresponding products. DS and HS have a peak at about 2.08 and 2.05 ppm respectively.

### DETAILED DESCRIPTION

### I. Abbreviations

API active pharmaceutical ingredient

CE capillary electrophoresis

DS dermatan sulfate

FDP final drug product

GAG glycosaminoglycan

H-NMR proton nuclear magnetic resonance

HS heparan sulfate

OS-CS oversulfated chondroitin sulfate

PCR polymerase chain reaction

RT-PCR reverse transcription polymerase chain reaction

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

cDNA (complementary DNA): A piece of DNA lacking internal, non-coding segments (introns) and transcriptional regulatory sequences. cDNA may also contain untranslated regions (UTRs) that are responsible for translational control in the corresponding RNA molecule. cDNA is usually synthesized in the laboratory by reverse transcription from messenger RNA extracted from cells.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached.

Unless otherwise specified, any reference to a DNA molecule is intended to include the complementary sequence of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its complementary strand. For instance, it is appropriate to generate probes or primers from the complementary sequence of the disclosed nucleic acid molecules.

Glycosaminoglycan (GAG): Polysaccharide composed of disaccharide subunits of N-acetyl-hexosamine and hexose or hexuronic acid, with varying degrees of sulfation occurring on each subunit. GAGs include heparin, heparin sulfate, chondroitin sulfate, dermatan sulfate, and heparan sulfate.

Heparinase: Family of enzymes that selectively cleaves the glycosidic linkage between the glucosamine and uronic acid components of the heparin polymer yielding oligosaccharide products. Heparinase I and II are active against both heparin and heparan sulfate. Heparinase I is approximately three times more active against heparin than heparan sulfate, cleaving at the hexosamine and O-sulfated iduronic acid bond. Heparinase II is approximately twice more active against heparan sulfate than heparin, cleaving at the 1-4 bond between hexosamine and either uronic or iduronic acid. Heparinases are widely available from commercial suppliers, for example, Sigma-Aldrich (St. Louis, Mo.). The unit of enzymatic activity (that is, one unit (U) of heparinase) is defined variously by different commercial suppliers; however, such definitions are in relation to the International Unit of heparinase where one unit of enzyme will form 1 μmole of unsaturated uronic acid per minute.

In vitro amplification: Techniques that increase the number of copies of a nucleic acid molecule in a sample or specimen. An example of in vitro amplification is the polymerase chain reaction (PCR), in which a pair of oligonucleotide primers is added to a sample under conditions that allow for the hybridization of the primers to a nucleic acid template in the sample. The primers are extended under suitable conditions, dissociated from the template, and then re-annealed, extended, and dissociated to amplify the number of copies of the nucleic acid. Other examples of In vitro amplification include, but are not limited to, RT-PCR, quantitative real time PCR, DNA replication, RNA transcription, and primer extension.

Nucleic acid polymerase: Any enzyme that catalyzes the synthesis of a polynucleotide through formation of a phosphodiester bond to join nucleotides into a nucleic acid polymer utilizing double stranded DNA, single stranded DNA, double stranded RNA or single stranded RNA as a template. Depending on the template, substrate nucleotide, and product polynucleotide, the polymerase can be a DNA dependent DNA polymerase, DNA dependent RNA polymerase, RNA dependent DNA polymerase, or RNA dependent RNA polymerase.

Oversulfated glycosaminoglycan: A glycosaminoglycan in which free hydroxyl groups are replaced by sulfate groups, which inhibits the activity of a nucleic acid polymerase, and which is resistant to degradation by heparinase. In particular examples of oversulfated glycosaminoglycan, all free hydroxyl groups are replaced by sulfate groups. Such examples include, but are not limited to, oversulfated chondroitin sulfate, oversulfated dermatan sulfate, oversulfated heparan sulfate, and oversulfated heparin sulfate. In other examples, the GAG is more highly, though not completely, sulfated, such as in chondroitin sulfate E.

Preparation: Any sample of any material that may be screened for oversulfated GAGs as described herein. In some examples, the preparation comprises heparin intended for medical therapeutic use (for instance in embodiments where the preparation is treated with heparinase). In other examples it is a sample of heparin eluted from or otherwise removed from a heparin-coated or impregnated device, such as a catheter, stent, or syringe or other medical device.

Quantitative real time PCR (qRT-PCR): A method for detecting and measuring products generated during each cycle of a PCR, which products are proportionate to the amount of template nucleic acid present prior to the start of PCR. The information obtained, such as an amplification curve, can be used to quantitate the initial amounts of template nucleic acid sequence. This information can also be used to detect or quantify the inhibition of nucleic acid polymerase activity, for instance inhibition due to the presence of heparin or an oversulfated GAG.

Reverse-transcription PCR (RT-PCR): A method for detecting, quantifying, or utilizing RNA present in a sample by a procedure wherein the RNA serves as a template for the synthesis of cDNA by a reverse transcriptase followed by PCR or quantitative real time PCR (qRT-PCR) to amplify the cDNA. It can also be used to detect the inhibition of nucleic acid polymerase activity due to the presence of heparin or an oversulfated GAG in a preparation, as well as to quantify the amount of oversulfated contaminant in a preparation.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

At least some of the material presented herein was published in Tami et al. (Biomaterials, 29:4808-4814, 2008), which is hereby incorporated by reference in its entirety.

### III. Overview of Several Embodiments

Disclosed herein are methods to detect and optionally quantify oversulfated glycosaminoglycan in a preparation, based on the inhibition of nucleic acid polymerase activity and in some instances resistance to enzymatic degradation.

In one embodiment, the method is detecting and/or quantifying oversulfated glycosaminoglycan in a medical preparation. For instance, examples of such methods comprise determining whether the medical preparation reduces or inhibits the activity of a nucleic acid polymerase. Such a reduction of activity would be indicative of the presence of glycosaminoglycan.

In another embodiment, the method is determining whether oversulfated glycosaminoglycan is present in a preparation containing heparin. Such methods involve treating a sample of the preparation with sufficient heparinase to substantially degrade any heparin therein, thereby producing a heparinase-treated sample; assaying activity of a nucleic acid polymerase in the presence of at least a portion of the heparinase-treated sample; and comparing the activity of the nucleic acid polymerase in the presence of the heparinase-treated sample to the activity of the nucleic acid polymerase in the absence of the heparinase-treated sample. In such examples, a measurable reduction of the nucleic acid polymerase activity in the presence of heparinase-treated sample indicates that oversulfated glycosaminoglycan is present in the preparation.

In another embodiment, the method is determining the relative quantity of an oversulfated glycosaminoglycan that is present in a preparation containing heparin. Such methods involve treating a sample of the preparation with sufficient heparinase to substantially degrade any heparin therein, thereby producing a heparinase-treated sample; assaying activity of a nucleic acid polymerase in the presence of increasing dilutions of the heparinase-treated sample; and comparing the activity of the nucleic acid polymerase in the presence of the heparinase-treated sample to activity of the nucleic acid polymerase in the presence of known quantities of oversulfated glycosaminoglycan. In such example methods, a similar reduction in the nucleic acid polymerase activity in the heparinase-treated sample and the nucleic acid polymerase activity in one of the known quantities of oversulfated glycosaminoglycan indicates the relative quantity of glycosaminoglycan in the preparation.

In various embodiments of these methods, the preparation is a therapeutic medical preparation. In various embodiments, the preparation is a heparin preparation.

In another embodiment, the preparation is from a medical device, for instance, it is from a medical device containing or coated with heparin. By way of example, the preparation in some instances is produced by removing (e.g., scraping, eluting, or otherwise removing) heparin from a medical device containing or coated with heparin.

In some embodiments of the described methods where the activity of a nucleic acid polymerase is assayed, the nucleic acid polymerase is a thermal stable DNA dependent DNA polymerase. By way of non-limiting example, one such nucleic acid polymerase is Taq polymerase. It is contemplated that assaying nucleic acid polymerase activity in some cases comprises running an in vitro nucleic acid amplification reaction, such as running a PCR amplification reaction, running a RT-PCR amplification reaction, or running a quantitative real time PCR amplification reaction.

In a further embodiment of the methods of detecting the presence or relative quantity of an oversulfated glycosaminoglycan contaminant in a heparin preparation, the nucleic acid polymerase activity in the presence of the heparinase-treated sample is reduced by a statistically significant amount of at least one standard deviation from the nucleic acid polymerase activity in the absence of the heparinase-treated sample. In another example of this embodiment, it is reduced by a statistically significant amount of at least three standard deviations.

In some of the described methods, the oversulfated contaminant comprises at least one oversulfated glycosaminoglycan of natural or synthetic origin. For instance, the oversulfated contaminant in some instances comprises oversulfated chondroitin sulfate, oversulfated heparan sulfate, oversulfated dermatan sulfate, or two or more thereof.

The methods provided herein employ, in various embodiments, heparinase I, heparinase II, or a mixture thereof.

Also provided are kits for carrying out the described methods. By way of example, a kit for use in a method of detecting the presence (and/or relative quantity) of an oversulfated glycosaminoglycan contaminant includes a container containing heparinase, nucleic acids (usually in a separate container), a nucleic acid polymerase (usually in a separate container), and instructions for comparing results of the method with a standard to provide a conclusion about the presence or quantity of oversulfated glycosaminoglycan contamination in a preparation.

### IV. Detection and/or Quantification of Oversulfated Compounds

Heparin and low molecular weight heparins are extensively used in the treatment of a wide range of diseases in addition to their classic anticoagulant activity, and can be found coating medical devices such as catheters, stents, and filters. Early in 2008, a sharp increase in heparin-associated severe adverse events, including over 80 deaths, was linked to the presence of a contaminant identified as oversulfated chondroitin sulfate (OS-CS). OS-CS is one of several oversulfated glycosaminoglycans (GAGs) of different origins that can potentially cause similar clinical problems underscoring the need to develop robust screening methods for contaminants in existing and future lots of heparin.

Currently, the FDA mandates screening heparin preparations for OS-CS contamination using proton-NMR (H-NMR) and capillary electrophoresis (CE). These methods require specialized machinery and significant concentrations of starting material, making these methods insufficient for testing samples of low heparin concentration. For example, testing of heparin flush-lock syringes (100 U/ml) by CE or H-NMR requires extensive sample preparation, including pooling material from about 20 syringes, evaporation, resuspension, and desalting of the collected sample. Likewise, these methods are not practical for evaluating contamination of heparin-coated devices, since the collection of heparin from those devices does not render enough material to be analyzed by CE or NMR methods without similar pooling and concentrating of samples.

As demonstrated herein, oversulfated GAGs inhibit the activity of nucleic acid polymerases, such as the Taq polymerase used for real-time PCR. Based on this finding, a simple, rapid, sensitive, and optionally high throughput screening method has been developed to detect and quantify oversulfated chondroitin sulfate (OS-CS) and other oversulfated GAG contaminants in any preparation including commercial lots of heparin, heparin derived from coated medical devices, and individual aliquots of low heparin concentration.

The results obtained using this method show a high level of correlation with existing methods such as H-NMR and CE, but also demonstrate that it is more sensitive and requires less starting material than the existing methods. The disclosed method requires less than 100 milliUnits (mU) of heparin as starting material, therefore avoiding the need to lyophilize and concentrate samples. Additionally, as shown in the examples below, this method can reveal the presence of OS-CS (Limit of quantitation: 500 pg or 0.16% w/w) and other oversulfated glycosaminoglycans (Limit of quantitation: 2.7 ng) in as little as 0.614 and/or 100 mU of heparinase-treated heparin, as opposed to the higher amounts of heparin required for CE and H-NMR (approximately 2 mg and 7 mg, respectively) and the lower limits of detection for OS-CS (1% for CE and 0.3-0.5% for H-NMR). In addition, assessment of polymerase inhibition allows for the amount of contaminant to be quantified by using known amounts of OS-CS as standard controls and titering down the heparin concentration to determine the lowest concentration that blocks gene amplification. Such titration simultaneously allows for quantification as well as optimization by the individual user for the particular experimental conditions unique to the individual laboratory.

Therefore, in addition to being a simple, rapid, sensitive, and high-throughput alternative to H-NMR and CE to screen commercial heparin preparations, the low requirement for starting material makes the herein described polymerase inhibition method a viable method for testing smaller quantities of heparin, as well as preparations retrieved from heparin coated devices such as syringes and tubing. Moreover, the ability to adapt the provided method to available starting material, nucleic acid polymerase used, and nucleic acid template sequences to test, allows it to be easily optimized for the requirements and experimental conditions of the individual user.

### V. Methods to Detect Oversulfated GAG Contaminants in a Preparation

### A. Screening Assay

Disclosed herein are methods of screening a heparin preparation for an oversulfated glycosaminoglycan (GAG) contaminant. These methods are based on the finding that oversulfated GAGs simultaneously inhibit the activity of a nucleic acid polymerase and are resistant to enzymatic degradation by heparinase.

Heparin inhibits the function of a wide variety of nucleic acid polymerases. In a general embodiment, a sample from any given heparin preparation is treated with heparinase to produce a heparinase-treated sample. This treatment can be under any conditions suitable for enzymatic degradation of heparin, most commonly at 25° C. for 2 hours. In oversulfated GAG contaminant-free samples, heparinase treatment measurably restores heparin-inhibited polymerase activity, for example in a PCR reaction. But in the presence of an oversulfated GAG contaminant, polymerase activity will remain inhibited to some measurable degree. Therefore, oversulfated GAG contamination of a preparation is detected by comparing polymerase activity of both heparinase-treated and non-heparinase-treated control samples from the starting preparation. Additionally, the oversulfated GAG contaminant can be quantified by simultaneous comparison of polymerase activity using samples of known heparin and oversulfated GAG concentrations.

In one embodiment of the screening assay, a sample from a heparin preparation is treated with heparinase I to degrade the heparin present in the sample to produce a heparinase-treated sample. The heparinase-treated sample (sample to be tested), a sample free of heparin or oversulfated GAG (100% polymerase activity), and one or more samples containing known concentration of oversulfated GAG (positive control) are then separately used in Taqman® quantitative real time PCR to amplify template DNA. The presence of oversulfated GAG is detected as a significant reduction in Taq polymerase activity as compared with the contaminant-free control sample. For quantification of oversulfated GAGs, the samples will be titered to determine the highest dilution that inhibits the polymerase.

### B. Preparation to be Assayed

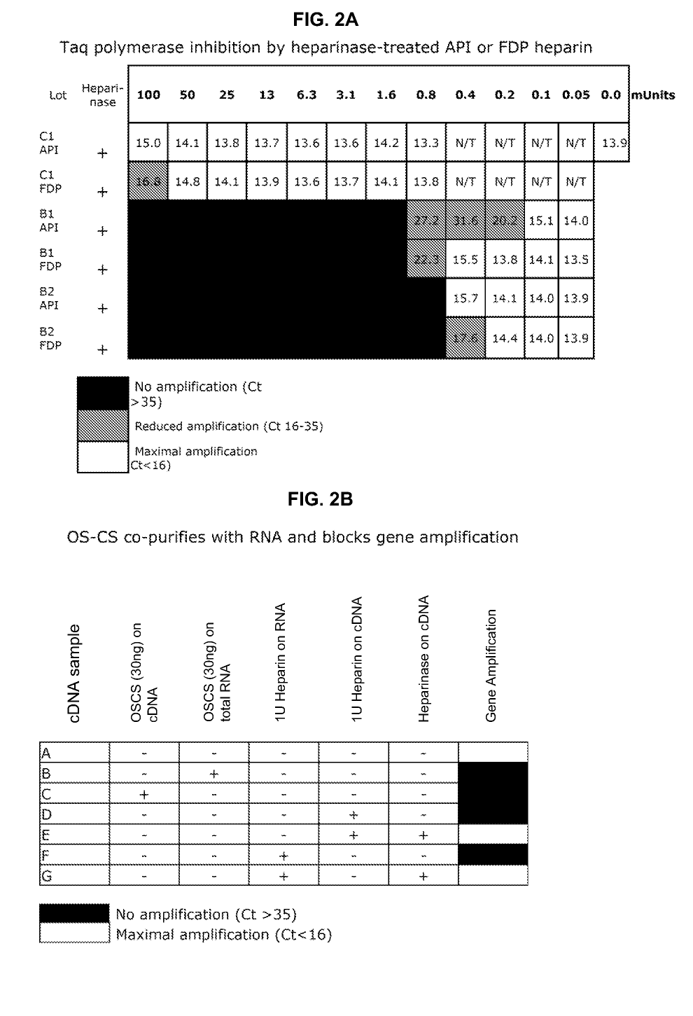

The preparation to be assayed can be any soluble material to be used in a nucleic acid polymerization reaction. In particular examples, the preparation is of a therapeutic agent for human and animal administration. In further examples, this agent is a heparin preparation suitable for heparinase digestion that may be further used in a nucleic acid polymerization reaction. Notably, this preparation of heparin can be either finished formulated final drug product (FDP) or the corresponding non-formulated active pharmaceutical ingredient (API). FIG. 2A shows that addition of as little as 1.6 mU of heparinase-treated heparin to the Taqman® reaction was sufficient to clearly differentiate between contaminated and uncontaminated lots regardless of whether the starting material was FDP or API.

In some examples, the heparin preparation is intended for use as an anticoagulant in multiple therapeutic settings including invasive surgical and dialysis procedures, deep venous thrombosis treatment, atrial fibrillation, or other coronary condition such as myocardial infarction. In other examples, the heparin preparation is used as part of a treatment for adult respiratory distress syndrome, allergic encephalomyelitis, allergic rhinitis, arthritis, asthma, cancer, and inflammatory bowel disease.

In other examples the heparin preparation is used as part of a pre-treatment or coating of materials that will be used in human or animal medical treatment. Such a preparation may also be intended for clinical research of therapies for human or animal disorders. In other examples the heparin is derived from materials and medical devices that have been coated with heparin, including stents, catheters, and syringes. In such examples, the heparin is derived from the material in any way such that the resultant preparation remains viable for the assay of nucleic acid polymerase activity. In some examples the heparin is released from the coating by breaking the covalent linkage anchoring the heparin to the coating. In other examples, where the heparin is a constituent of a drug-release type antithrombotic coating, heparin is released in the manner appropriate to the coating including eluting the attached heparin in an aqueous solvent. In further examples, where the heparin is complexed with a hydrogel, the heparin is released by exposure to an ionic solution, or to an increase in temperature.

In other examples, the heparin preparation is taken from a pre-filled sample including flush-lock heparin syringes.

One of ordinary skill in the art will understand other circumstances in which it would be beneficial to determine if (or to what extent) a heparin preparation is contaminated with GAGs.

### C. Oversulfated Compounds

The oversulfated GAG to be assayed for by the methods disclosed herein is any GAG that contains more sulfate groups at its available nitrogen and oxygen positions than usually found in nature, inhibits the activity of a nucleic acid polymerase, and is resistant to degradation by heparinase. With these combined characteristics, the activity of a nucleic acid polymerase in a contaminated preparation will remain measurably inhibited after removal of heparin by heparinase treatment. But in contaminant-free preparations, any inhibited nucleic acid polymerase activity will be restored after heparinase treatment.

Notably, while naturally occurring GAGs such as dermatan sulfate or heparan sulfate may be partially sulfated to varying degrees, both require higher sulfate concentrations to block Taq polymerase activity, reducing the concerns for false positive results.

In particular examples, the oversulfated GAG is naturally occurring. In other examples the oversulfated GAG is synthetic. In particular examples, the oversulfated GAG is oversulfated chondroitin sulfate. In further examples the oversulfated GAG is oversulfated heparan sulfate. In other examples the oversulfated GAG is oversulfated dermatan sulfate. In other examples the oversulfated GAG is chondroitin sulfate E or A.

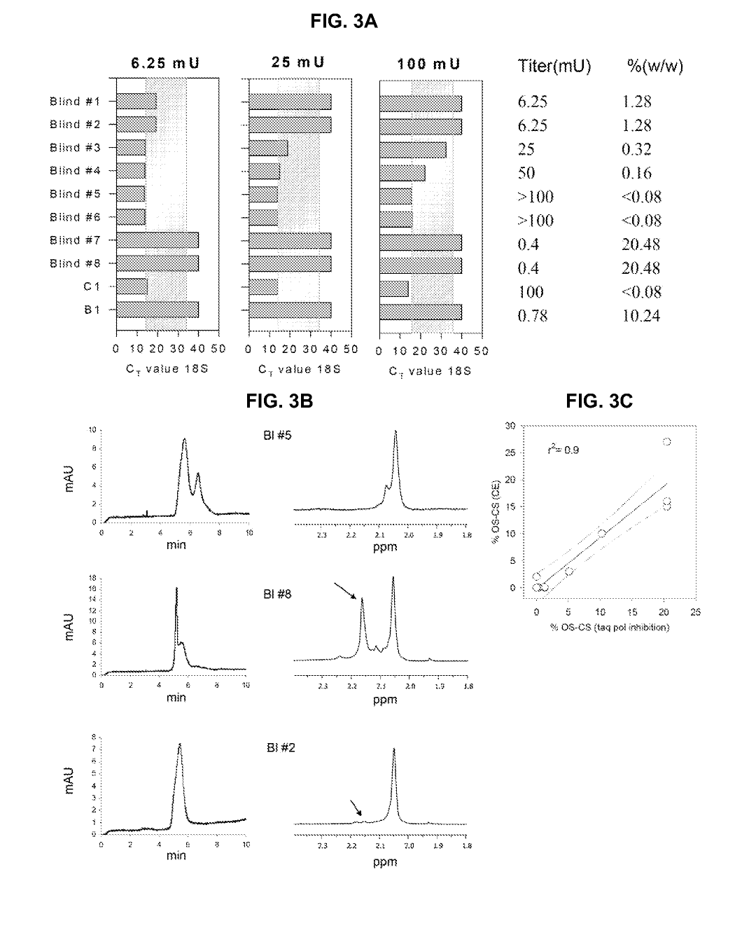

In particular examples, the detection of oversulfated GAG contaminant by the disclosed methods can be confirmed by CE and H-NMR spectra. As shown in FIG. 3B (right), H-NMR spectra generated from such samples demonstrate that for the N-acetyl region of H-NMR, the heparin signals at 2.04 ppm, dermatan sulfate at 2.08 ppm and OS-CS at 2.16 ppm are as previously described (Guerrini et al., Nat. Biotech., 26: 669-675, 2008). As additionally shown in FIG. 3B (left), CE shows a sharp peak for the contaminant before heparin corresponding to dermatan sulfate (2.08 ppm). In a different contaminated sample, contaminant is confirmed by CE as a sharp peak after the heparin and confirmed by H-NMR with a proton peak at 2.16 ppm (arrows) in the N-acetyl region.

### D. Heparinase Treatment

The heparinase used in the described methods is any heparinase that degrades heparin but is substantially ineffective against oversulfated GAGs. The conditions of heparinase digestion, including the amount of heparinase required, are such that the heparinase substantially degrades the heparin in a given preparation. Such degradation of known polymerase-inhibitory quantities of heparin in control samples is sufficient to allow a distinction between inhibited and functional nucleic acid polymerase activity. In some examples, less than 50%, 40%, 30%, 20%, 10% or even less than 5% of the original concentration of heparin remains in the preparation after it has been substantially degraded.

In other particular examples, the heparinase used is heparinase I. In other examples, the heparinase used is heparinase II. Heparinase I and II are active against both heparin and heparan sulfate. Heparinase I is approximately three times more active against heparin than heparan sulfate, cleaving at the hexosamine and O-sulfated iduronic acid bond. Heparinase II is approximately twice more active against heparan sulfate than heparin, cleaving at the 1-4 bond between hexosamine and either uronic or iduronic acid (Desai et al., Arch. of Biochem. and Biophys., 306: 461-468, 1993; Ernst et al., Crit. Rev. in Biochem. and Molecular Biol., 30: 387-444, 1995). Heparinases are widely available from commercial suppliers, for example, Sigma-Aldrich (St. Louis, Mo.).

In particular examples, the amount of heparinase I used is 6.5 mU, 25 mU, 100 mU, or 1 U of enzyme, for instance about 0.8 to 1.2 Units of enzyme for each unit of heparin, or particularly about 1 U of enzyme for each unit of heparin. The Unit definitions of both heparin and heparinase are well known to those of skill in the art. A Unit of heparin (or “Howell Unit”) is the amount of heparin needed to keep 1 ml of cat's blood fluid for 24 hours at 0° C. This is equivalent to about 0.002 mg of pure heparin. A Unit of heparinase is the amount needed to form 1 μmole of unsaturated uronic acid per minute.

In further examples, the samples to be assayed are treated with heparinase I (1:1 unit:unit reaction) for 2 hours at 25° C. in a buffer containing 4 mM Tris-HCl pH 7.5, 0.8 mM CaCl, 10 mM NaCl and 20 U of RNase inhibitor (ABI). After treatment, the samples are diluted to a final volume of 50 μl and serial dilutions prepared in DEPC water, as specified, in preparation for nucleic acid synthesis by a nucleic acid polymerase.

In further examples, such as in the screening of any therapeutic agent, the samples to be assayed are not known to contain heparin. In such an example, the disclosed method is practiced as described, but the heparinase treatment step is optional. It could be used as described to remove any trace amounts of heparin that might be in the preparation to be screened. However, most likely the assay would be based on the inhibition of polymerase activity by an oversulfated GAG contaminant and the comparison of polymerase activity between clean and potentially contaminated preparations.

### E. Nucleic Acid Polymerases

The nucleic acid polymerase used in the methods disclosed herein may be any nucleic acid polymerase that is inhibited both by heparin and by oversulfated GAG. Because oversulfated GAG is resistant to heparinase digestion, any heparin-inhibition of polymerase activity will be restored in heparinase-treated samples that contain heparin and are free from oversulfated GAG contamination, but will remain inhibited in oversulfated GAG-contaminated preparations.

In particular examples the nucleic acid polymerase is a DNA dependent DNA polymerase, DNA dependent RNA polymerase, RNA dependent DNA polymerase, or RNA dependent RNA polymerase.

Heparin inhibits DNA replication activity of a wide variety of DNA polymerases (Holodniy et al., J. Clin. Microbial., 29: 676-679, 1991; Furukawa & Bhavanandan, Biochim. Biophys. Acta 740: 466-475, 1983; Izraeli et al., Nucleic Acids Res., 19: 6051, 1991; Yokota et al., J. Clin. Lab. Anal., 13: 133-140, 1999). In particular examples the nucleic acid polymerase is a thermally stable DNA dependent DNA polymerase capable of amplifying DNA in a polymerase chain reaction. In further examples, the thermal stable DNA polymerase is the Taq polymerase, polymerase mixtures containing Taq, Pfu polymerase or polymerase mixtures containing Pfu (Yokota et al., J. Clin. Lab. Anal., 13: 133-140, 1999, Taylor A. C. Molecular Ecology, 6: 383-385, 1997; Mathur et al., Nucleic Acids Res., 19: 6952, 1991). In other examples the nucleic acid polymerase is a member of the DNA polymerase α or δ families of polymerases. In further examples the nucleic acid polymerase is a member of the E. coli pol II family of DNA polymerases (DiCioccio et al., Cancer Research, 38: 2401-2407, 1978; Goulian and Heard, Nucleic Acids Res., 18: 4791-4796, 1990).

Heparin also inhibits transcription by RNA polymerases. In particular examples the nucleic acid polymerase is a member of the E. coli RNA polymerase family of polymerases (Pfeffer et al., J. Biol. Chem., 252:5403-7, 1977). In other examples it is a bacteriophage RNA polymerase, such as the T7 RNA polymerase (Chamberlin and Ring, J. Biol. Chem., 248: 2245-2250, 1973). In other examples it is a member of the eukaryotic RNA polymerase II family (Kasdesch and Chamberlin, J. Biol. Chem., 257: 5286-5295, 1982).

Additionally, heparin inhibits reverse transcriptase activity. Thus, in further examples, the nucleic acid polymerase is Moloney murine leukemia virus (MMLV), human immunodeficiency virus (HIV), simian sarcoma virus (SSV), or similarly related reverse transcriptases (Holodniy et al., J. Clin. Microbiol., 29: 676-679, 1991).

### F. Determination of Nucleic Acid Polymerase Activity

The inhibition of a nucleic acid polymerase by an oversulfated GAG contaminant may be measured in any way available to detect activity of the specific polymerase. Thus, in particular examples utilizing a DNA polymerase to screen for the oversulfated GAG contaminant, DNA synthesis activity would be measured. Likewise in a screen utilizing a RNA polymerase to screen for the oversulfated GAG contaminant, RNA synthesis activity would be measured. In all examples, a measurable reduction in activity is any statistically significant reduction in polymerase activity compared to polymerase activity assayed in the absence of the heparinase-treated sample. This reduction in activity can be as little as one standard deviation from that of the activity of control samples. Polymerase activity can be visualized or detected by any means including photographic, spectroscopic, or radioactive methods.

In particular examples using quantitative real time PCR to assay for oversulfated GAG contaminant, polymerase activity is directly measured by detection of a fluorescent signal generated by amplification product. In other examples, where the inhibition of RNA polymerase is used to screen for contamination, polymerase activity can be indirectly measured by RT-PCR.

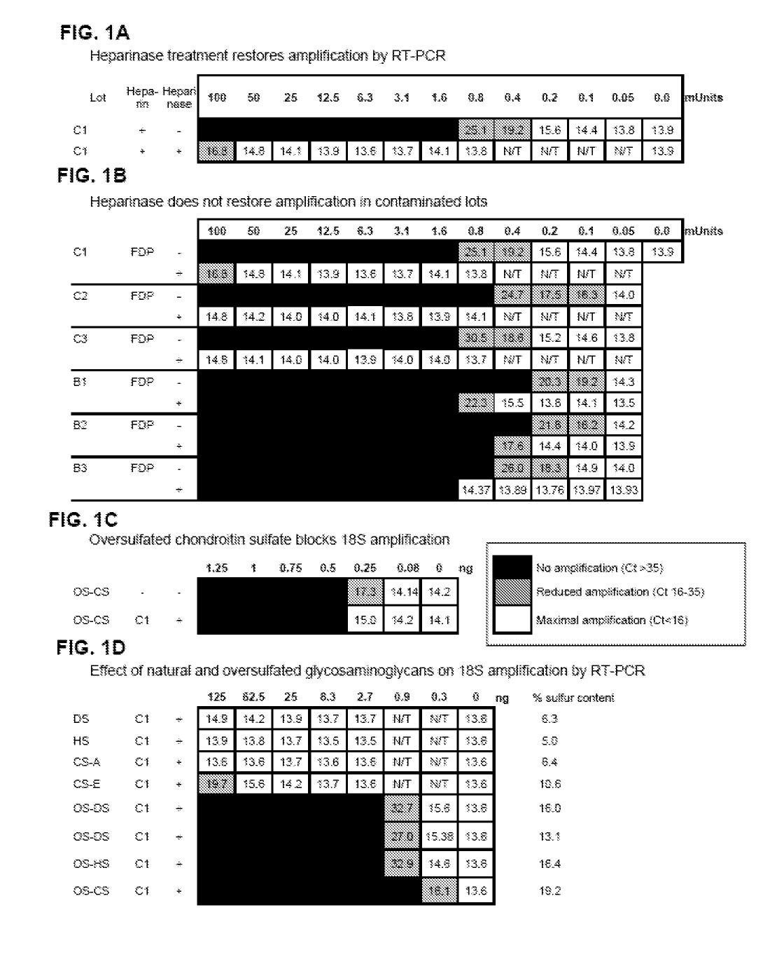

In other particular examples, quantitative real time PCR is used to screen for the oversulfated GAG contaminant. In such an example, each test or control sample can be screened using the Taqman® Gene Expression assay for human 18S rRNA (20×) and 2× Universal PCR master mix (ABI) in a 25 μl reaction. As shown in FIGS. 1 and 2, amplification levels of 18S may be expressed as C_{T} values. C_{T} values represent the cycle at which amplification of a target gene is first detected. Inhibition of polymerase activity is determined relative to the C_{T} value of a control, such as a heparinase treated sample, or sample(s) with defined amounts of inhibiting compound. Although the ribosomal 18S rRNA is used as the assay template sequence in this example, any RNA or DNA sequence would also be a suitable template sequence to test for nucleic acid polymerase activity. This is shown in Table 1, which compares the effects of increasing concentrations of oversulfated GAGs on Taq polymerase amplification of 18S, GAPDH, and Actin B template sequences, and demonstrates that the methods provided herein are not specific for specific nucleic acid templates. Thus, any template can be used in the methods disclosed herein.

In further examples, the inhibitory effects on PCR, RT-PCR, or other in vitro nucleic acid amplification reactions may be used to detect the presence of oversulfated GAG contaminant. In these methods, such inhibitory effects may be detected by any means available to detect the presence or absence of DNA or RNA synthesis, including gel electrophoresis.

### VI. Methods to Quantify Oversulfated GAG Contaminants in a Preparation

Also disclosed herein are methods to quantify oversulfated GAG compounds in a preparation. Such a contaminant can be quantified employing the same methods and variations disclosed for the detection of oversulfated GAG, with the additional step of comparing the nucleic acid polymerase activity in the presence of the heparinase-treated sample to nucleic acid polymerase activity in the presence of known quantities of oversulfated glycosaminoglycan. Using such a comparison, a correlation between the reduction in the nucleic acid polymerase activity in the heparinase-treated sample and the nucleic acid polymerase activity in one of the known quantities of oversulfated glycosaminoglycan indicates the relative quantity of glycosaminoglycan in the preparation.

The known quantities of oversulfated GAG are produced from a serial dilution of a known concentration of material. The dilutions are then used as controls to calibrate polymerase activity under the particular assay conditions.

In a particular example, quantitation of the oversulfated GAGs is performed by establishing a cutoff at the minimum concentration of synthetic OS-CS that completely blocks Taq polymerase and titering all the samples until the activity of Taq polymerase was restored. In a further particular example, this cutoff occurs at 500 pg of OS-CS. In such an example, the maximal dilution that completely blocks the enzyme is then assumed to have at least 500 pg of contaminant.

### VII. High Throughput Detection and Quantification of Oversulfated GAG Contaminant

The disclosed methods for detection and quantification of oversulfated GAG in a preparation can be used to screen a large number of samples simultaneously (that is, can be used in high-throughput applications). In particular examples, such high-throughput screening is accomplished through detecting oversulfated GAG polymerase-inhibition in the context of PCR, RT-PCR, or quantitative real time PCR. In particular examples, this method is carried out by any of the numerous methods to process multiple amplification reactions. Such methods include, but are not limited to, amplification in a thermal cycler compatible with a microtiter plate, use of an automated liquid handling and thermal cycler workstation, or other high throughput techniques such as that according to Morrison et al. (Nucl. Acids Res., 34: e123, 2006).

### VIII. Kits

This disclosure also provides kits that enable a user to screen for oversulfated contaminants of heparin preparations. Such kits would contain at least but would not be limited to containers of heparinase, a particular nucleic acid polymerase, nucleic acids, and instructions for carrying out the disclosed methods of oversulfated GAG contaminant detection. In particular examples, the polymerase would be a DNA polymerase. In other particular, examples the polymerase would be a RNA polymerase.

Certain kits can include Taq polymerase and reagents necessary for quantitative real time PCR, including but not limited to, nucleic acid template, amplification primers, one or more fluorescent labels for detection of the amplified template, nucleotides, and buffers necessary to carry out quantitative real time PCR. In further examples, such kits can also contain reverse transcriptase and reagents necessary to reverse transcribe and RNA template in preparation for RT-PCR.

Certain kits may also contain oversulfated GAG of known quantity, for instance for use as positive control samples, quantitation of oversulfated GAG contaminant, or optimization of the screening assay.

The materials provided in such kits may be provided in any form practicable, such as suspended in an aqueous solution or as a freeze-dried or lyophilized powder, for instance. Kits according to this invention can also include instructions, usually written instructions, to assist the user in carrying out the detection and quantification methods disclosed herein. Such instructions can optionally be provided on a computer readable medium or as a link to an internet page.

The container(s) in which the reagents are supplied can be any conventional container that is capable of holding the supplied form, for instance, microfuge tubes, ampoules, or bottles. In some applications, the heparinase, nucleic acid polymerase, or reagent mixtures required for in vitro nucleic acid amplification may be provided in pre-measured single use amounts in individual, typically disposable, tubes, microtiter plates, or equivalent containers. The containers may also be compatible with a specific automated liquid handling apparatus.

The amount of a reagent supplied in the kit can be any appropriate amount, depending for instance on the market to which the product is directed. For instance, if the kit is adapted for research or clinical use, the amount of each reagent, such as heparinase or nucleic acid polymerase, would likely be an amount sufficient for multiple screening assays. In other examples where the kit is intended for high throughput industrial use, the amounts could be sufficiently increased to accommodate multiple hundreds of assays.

The following examples are provided to illustrate certain particular features and/or embodiments. These examples should not be construed to limit the invention to the particular features or embodiments described.

### EXAMPLES

### Example 1

### Inhibition of Taq Polymerase as a Method for Screening Heparin for Oversulfated Contaminant

This example shows the effects of oversulfated GAG contaminants on Taq polymerase activity, demonstrates the use of the disclosed method to detect and quantify the presence of the oversulfated GAG contaminant OS-CS, compares the disclosed method with current contaminant detection methods, and lastly shows the effects of oversulfated GAGs other than OS-CS on Taq polymerase activity.

### A. Materials and Methods

Samples and reagents: Heparin samples: Active pharmaceutical ingredient (API) and final drug product (FDP) for samples B1-3 and C1-3 were obtained by the FDA from Baxter Healthcare (1000 U/ml or 5000 U/ml in 10 ml and 30 ml vials). Eight blinded samples (blind #1-8) were obtained during the FDA's inspections. Chondroitin sulfate E was obtained from Seikagaku (Japan) and characterized by the H-NMR method stated below. Heparinase I was obtained from Sigma (St Louis, Mo.). RNA was extracted from MDA-MB-231 human breast cancer cell line grown in DMEM/F12 (50/50) media.

Heparinase treatment: Heparin was treated with heparinase I (1:1 unit:unit reaction unless otherwise noted) for 2 hours at 25° C. in a buffer containing 4 mM Tris-HCl pH 7.5, 0.8 mM CaCl, 10 mM NaCl, and 20 U of RNase inhibitor (ABI). After treatment, the heparin was diluted to a final volume of 50 μl and serial dilutions were prepared in DEPC water as specified. Where indicated, other GAGs and oversulfated GAGs (dermatan sulfate, chondroitin sulfate, and heparan sulfate) were treated with heparinase under the same conditions as described above.

Screening Assay: cDNA was generated using total RNA from MDA-MB-231 human breast cancer cell line extracted using Trizol reagent (Invitrogen, Carlsbad, Calif.) as per manufacturer's instructions. For each cDNA reaction 1 μg total RNA was reverse transcribed using High Capacity cDNA Reverse Transcription Kit (Applied Biosystems, Foster City Calif.) in a volume of 20 μl and further diluted in DEPC water to 100 μl final volume. The concentration of cDNA was determined using Quant-iT OliGreen ssDNA Reagent (Invitrogen). For each Taqman® reaction, 25 ng of cDNA in a 2.5 μl volume were mixed with 2.5 μl of each dilution of heparinase-treated heparin or hypersulfated GAGs and then subjected to real time PCR using Taqman® Gene Expression assay for human 18S rRNA (20×) and 2× Universal PCR master mix (ABI) in a 25 μl reaction. Amplification levels of 18S are expressed as C_{T} values. C_{T} values represent the cycle at which amplification of a target gene is first detected. The amplification was analyzed using manual settings with a threshold value of 0.1 and the SDS2.3 software from ABI. C_{T} values<16 cycles indicate there was no significant inhibition of the Taq polymerase activity while C_{T} values of >35 indicate complete inhibition of the assay. Quantification of the oversulfated GAGs was performed by establishing a cutoff at the minimum concentration of synthetic OS-CS that completely blocks Taq polymerase (500 pg) and tittering all the samples until the activity of Taq polymerase was restored. The maximal dilution that completely blocked the enzyme was then assumed to have at least 500 pg of contaminant.

H-NMR analysis: All samples were analyzed using a Varian 500 MHz Inova instrument. Samples were prepared by dissolving approximately 10 mg of sample in 0.6 ml of deutered water spiked with a reference compound TSP (tri-methyl-silyl propionate-, sodium salt). The reference TSP signal is set to 0.00 ppm, which is referenced at 0.00 ppm). Samples were run at 25° C. Spectral parameters include no less than 16 transients, 90 degree pulse width, acquisition time of at least one second, time between transients of 20 seconds and a spectral window of 8000 hz.

Capillary Electrophoresis: CE was conducted on a Hewlett Packard 3D-CE instrument equipped with a diode array detector set at a wavelength of 200 nm (band width 10 nm). Separations were performed in a bare fused silica capillary, internal diameter 50 μm, 64.5 cm-total length, 56 cm-effective length with a column temperature of 25° C. The polarity was negative with a voltage of 30 kV. Samples were dissolved in Milli-Q water at a concentration of approximately 10 mg/mL and filtered through 0.2 μm cellulose acetate membrane filters (Micro-Spin filter tubes, Alltech Associates, Deerfield, Ill., USA). The sample solutions were injected using hydrodynamic pressure at 50 mbar for 10 seconds. The electrolyte solution was 36 mM phosphate buffer (pH 3.5) filtered with a 0.2 μm cellulose acetate syringe filter (Grace, Deerfield, Ill.). The capillary column was preconditioned at the beginning of each day by flushing with 1M NaOH, 0.1M NaOH, and water, each for 2 min, and prior to running each sample by flushing with water for 2 mM and electrolyte solution for 2 mM Monobasic sodium phosphate, monohydrate, ACS grade, and phosphoric acid 85%, N.F. Food Grade, were obtained from Mallinckrodt Baker, Inc (Phillipsburg, N.J., USA). 1M and 0.1M Sodium hydroxide solutions for High Performance Capillary Electrophoresis (HPCE) were from Hewlett Packard (Waldbronn, Germany).

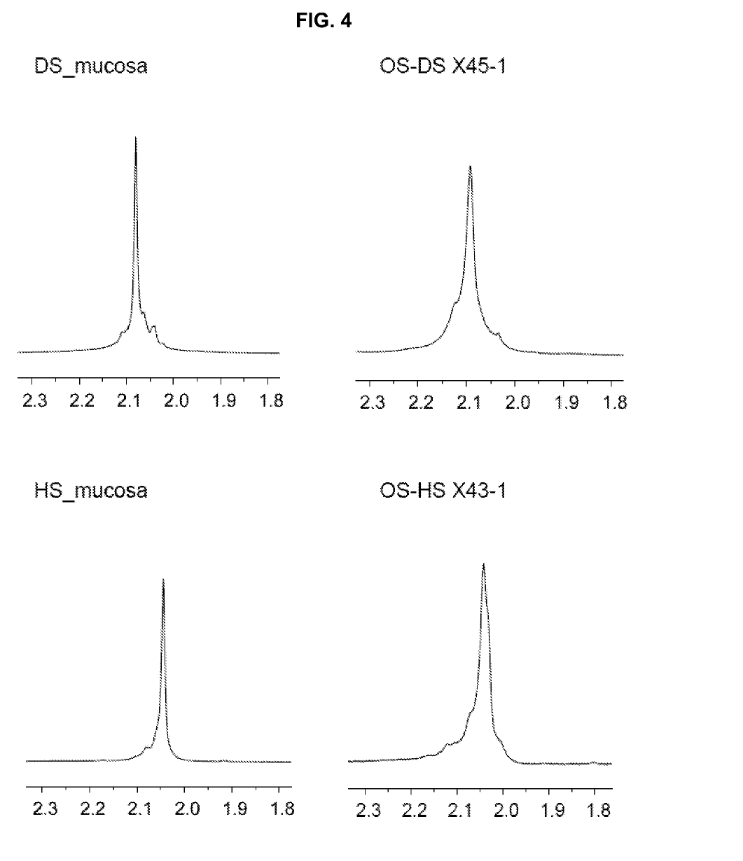

Chemical sulfonation of chondroitin sulfate: Fully sulfated chondroitin sulfate was prepared from chondroitin sulfate as described (Maruyama et al., Carbohydrate Research, 306: 35-43, 1998). Thus, 139 mg chondroitin sulfate tributylamine salt and 1.2 g sulfur trioxide pyridine complex were dissolved in 2 ml dry N,N-dimethylformamide and heated for 1 h at 40° C. The reaction solution was adjusted to pH 9 with 1M NaOH and diluted with 3 volumes of ethanol saturated with sodium sulfate, generating a precipitate. The mixture was cooled in a refrigerator, centrifuged, and the solid material was purified by dialysis and lyophilization. Heparan sulfate, dermatan sulfate, chondroitin sulfate A, and E as well as over-sulfated heparin (OS-HS) and dermatan sulfate (OS-DS) were synthesized in house as described (Chen et al., J. Biol. Chem., 280: 42817-42825, 2005; Nadkarni et al., Carbohydrate Research, 290: 87-96, 1996) and characterized by H-NMR (FIG. 4), CE and elemental analysis for sulfur content (performed at Galbraith Laboratories).

### B. Results

### Effect of Heparin Contaminants on Gene Amplification by Taqman® PCR

Real time-PCR (Taqman® PCR) is a highly sensitive method for detecting changes in gene expression. Gene amplification depends on a thermostable DNA polymerase from Thermus aquaticus (Taq pol). Previous studies have shown that heparin competitively inhibits several different cellular DNA polymerases including Taq polymerase (Holodniy et al., J. Clin. Microbiol., 29: 676-679, 1991; Furukawa & Bhavanandan, Biochim. Biophys. Acta 740: 466-475, 1983; Izraeli et al., Nucleic Acids Res., 19: 6051, 1991; Yokota et al. J. Clin. Lab. Anal., 13: 133-140, 1999), but this inhibition can be overcome by the use of heparinase (Izraeli et al., Nucleic Acids Res., 19: 6051, 1991; Johnson et al., Biotechniques, 35: 1140-1144, 2003). Shown in FIG. 1 are the levels of 18S amplification from 25 ng cDNA derived from MDA-MB-231 cells as determined by Taqman® PCR. While the 18S rRNA is used here, any RNA or DNA template is suitable. Addition of progressively higher amounts of heparin induces a corresponding reduction in Taq polymerase activity that can be monitored by assessing 18S cDNA amplification. Cycle thresholds (C_{T}) <16 indicate no inhibition of the Taq polymerase activity whereas C_{T} values>35 denote complete inhibition of the assay. Treatment of heparin with 1 unit of heparinase for 2 hrs at 25° C. overcomes the blocking activity of up to 50 mU of heparin (FIG. 1A).

To determine whether the presence of contaminants modulates the blocking effect of heparin on Taq pol, an assay was designed in which heparin from lots that were associated with adverse clinical effects (B1, B2 and B3) as well as from control lots with no visible clinical effects (C1, C2, C3) were treated with heparinase (or left untreated). Sequential dilutions of heparin were then added onto 25 ng of cDNA and 18S was amplified by Taqman® PCR. As shown in FIG. 1B, while 18S amplification was maximal in control heparinase treated lots (C1-3), heparinase treatment did not restore 18S amplification of cDNA exposed to lots of heparin associated with adverse events (B1-3). This suggested that a contaminant in the heparin inhibits the Taq polymerase enzyme. Of note, addition of as little as 6.25 mU of heparinase-treated heparin to the Taqman® reaction was sufficient to clearly differentiate between contaminated and uncontaminated lots regardless of whether the starting material was finished formulated drug product or the corresponding non-formulated active pharmaceutical ingredient (API) diluted in water (FIG. 2A).

### Use of Taq Polymerase Inhibition to Screen for the Presence of OS-CS in Heparin

OS-CS was identified as a contaminant present in lots of heparin that have been linked to adverse events. Persulphated chondroitin sulfate is not susceptible to heparinase I or II (or chondroitinase) degradation. To determine whether OS-CS directly inhibits Taq polymerase activity, decreasing concentrations of OS-CS were added to cDNA and amplified by Taqman® PCR. As shown in FIG. 1C, addition of 500 pg of OS-CS completely blocked Taq polymerase mediated 18S cDNA amplification.

In order to determine the minimum % (w/w) of contaminant that could be detected in heparin, a representative potency of heparin was used (6.25 μg/U). Since 500 pg of OS-CS completely block 18S amplification, the lowest titer of heparin that completely blocked amplification was assumed to contain at least 500 pg of OS-CS. From this, the limit of detection was calculated as 0.16% (w/w), which is below the level of detection for both proton NMR and CE. Importantly, the presence of heparin and heparinase did not modify the sensitivity of the assay (FIG. 1C). These results indicate that failure to amplify 18S cDNA can be used as a rapid and sensitive diagnostic test to screen for the presence of OS-CS in heparin. Lastly, similar gene amplification inhibition levels were evident whether the suspect heparin or the OS-CS were added to the cDNA or to the RNA used to generate the cDNA, indicating that any contaminant present likely co-purifies with nucleic acids (FIG. 2B). This raises the possibility that the presence of oversulfated contaminants in heparin may have interfered with PCR-based assays that are currently used to test heparin lots for contaminants from material from other natural sources such as bovine heparin.

Comparison of the Taq Polymerase Inhibition Method with Current Methods Used to Screen and Quantify OS-CS

To verify the disclosed method as a screening assay for the presence of oversulfated contaminants, 8 lots of heparin API were tested in a blinded manner. Each was treated with heparinase I, and titered into a Taqman® PCR reaction. Addition of 6.25 mU of heparin resulted in complete inhibition of amplification for two samples (Blind #7 and 8) (FIG. 3A). Those samples were later identified as having 15 and 27% OS-CS contamination by CE, and showing significant peaks at 2.16 ppm by H-NMR (FIG. 3B, right). For blind samples #1 and 2, there was reduced amplification of 18S at 6.25 mU and complete inhibition when 25 or more mU of heparin were added to the PCR reaction. Subsequent unblinding of the H-NMR and CE profiles for these particular samples showed the presence of a weak signal at 2.16 ppm by H-NMR but no visible peak by CE, a profile that suggests marginal OS-CS contamination (FIG. 3B, left). Samples 3 and 4 showed a reduced 18S amplification at heparin concentrations of 100 and 25 mU of heparin but did not completely block it suggesting that trace levels of contaminants could be present in these samples. This indicated that Taq polymerase inhibition is more sensitive to OS-CS contamination than CE or H-NMR. The remaining samples (Bl #5 and 6) did not inhibit 18S amplification. Lastly, comparison of the OS-CS concentration, as determined by inhibition of 18S amplification, to the percent OS-CS as determined by CE shows a high level of correlation (r²=0.9, p<0.001) confirming that the Taqman®-inhibition based method is an effective semi-quantitative screening assay, likely to identify very low levels of over-sulfated contaminants in heparin.

### Susceptibility of Taq Polymerase to Other Potential Oversulfated GAGs

Heparin is commonly extracted from porcine intestinal mucosa or bovine lung, and preparations may contain small amounts of other glycosaminoglycans. The presence of naturally occurring dermatan sulfate (DS), heparan sulfate (HS) or chondroitin sulfate A (CS-A), when spiked into heparin and treated with heparinase did not inhibit Taq polymerase activity at any of the concentrations tested (0-125 ng) (FIG. 1C). Chondroitin sulfate E (CS-E), which is more sulfated that chondroitin sulfate A (CS-A) or DS, did show a trace of inhibitory effect at the highest concentration tested (125 ng). In contrast, under the same conditions, all completely sulfated glycosaminoglycans tested, including oversulfated heparan sulfate (OS-HS), two different forms of oversulfated dermatan sulfate (OS-DS) and OS-CS significantly reduced 18S amplification. Further, the magnitude of the inhibitory effect on Taq polymerase correlated with the degree of sulfation of each compound as determined by elemental analysis (r²=−0.93, p<0.001). This indicates that Taq polymerase inhibition is a useful tool to screen heparin preparations for oversulfated GAG contaminants.

### Example 2

### The Inhibition of Taq Polymerase as a Method for Screening Heparin for Oversulfated Contaminant is not Restricted to a Particular DNA Sequence

This example illustrates that using inhibition of Taq polymerase to assay for oversulfated GAGs is not restricted to a particular cDNA template.

To compare the use of different template sequences to measure the inhibition of Taq polymerase activity by oversulfated GAGs, heparin preparations were processed and RNA was isolated as detailed in Example 1, except that cDNAs were generated and amplified from the Actin B and GAPDH RNA sequences, in addition to the 18S rRNA.

Table 1 compares the effects of different oversulfated GAGs on the amplification of 18S, Actin B, and GAPDH sequences by Taq polymerase, as shown by mean cycle thresholds (C_{T}) of amplification detection. The C_{T} value indicating no inhibition of Taq polymerase activity varied slightly by template, but was maximally <20. C_{T} values>35 denote complete inhibition of the assay. Addition of progressively higher amounts of over sulfated chondroitin sulfate (OS-CS), oversulfated dermatan sulfate (OS-DS), and oversulfated heparan sulfate (OS-HS) to cDNA induced a corresponding reduction in Taq polymerase amplification of 18S, Actin B, and GAPDH. In contrast, addition of heparan sulfate (HS) at comparable concentrations did not inhibit Taq polymerase amplification of any of the tested templates. Notably, while the amplification of all three template sequences was inhibited by oversulfated GAG, the use of different templates may change the sensitivity of the Taq inhibition assay. For example, GAPDH showed greater sensitivity to lower amounts of OS-CS and OS-DS than either 18S or Actin B.

TABLE 1
Inhibition of Taq polymerase is not template specific
A. Oversulfated Chondroitin Sulfate

| ng OS-CS | 0 | 0 (+3SD) | 0.08 | 0.25 | 0.5 | 0.75 | 1 | 1.25 |
|---|---|---|---|---|---|---|---|---|
| 18S | 13.81 | 14.56 | 13.81 | 14.38 | 18.89 | 31.20 | 40.00 | 40.00 |
| Actin B | 17.71 | 19.57 | 18.48 | 26.89 | 38.84 | 40.00 | 40.00 | 40.00 |
| GAPDH | 17.60 | 18.02 | 17.22 | 25.13 | 40.00 | 40.00 | 40.00 | 40.00 |

B. Oversulfated Dermatan Sulfate

| ng OS-DS | 0 | 0 (+3SD) | 0.3 | 0.9 | 2.7 | 8.3 | 25 |
|---|---|---|---|---|---|---|---|
| 18S | 13.81 | 14.56 | 14.13 | 18.29 | 40.00 | 40.00 | 40.00 |
| Actin B | 17.71 | 19.57 | 25.45 | 31.85 | 40.00 | 40.00 | 40.00 |
| GAPDH | 17.60 | 18.02 | 24.76 | 39.12 | 40.00 | 40.00 | 40.00 |

C. Oversulfated Heparan Sulfate

| ng OS-HS | 0 | 0 (+3SD) | 0.3 | 0.9 | 2.7 | 8.3 | 25 |
|---|---|---|---|---|---|---|---|
| 18S | 13.81 | 14.56 | 14.49 | 26.00 | 40.00 | 40.00 | 40.00 |
| Actin B | 17.71 | 19.57 | 27.12 | 40.00 | 40.00 | 40.00 | ND |
| GAPDH | 17.60 | 18.02 | 25.98 | 40.00 | 40.00 | 40.00 | 40.00 |

D. Heparan Sulfate

| ng HS | 0 | 0 (+3SD) | 8.3 | 25 | 62.5 | 125 | 250 |
|---|---|---|---|---|---|---|---|
| 18S | 13.81 | 14.56 | 13.62 | 13.66 | 13.69 | 13.96 | 14.36 |
| Actin B | 17.71 | 19.57 | ND | 18.36 | ND | ND | ND |
| GAPDH | 17.60 | 18.02 | 17.68 | 18.37 | ND | ND | ND |

These results demonstrate that oversulfated GAG contaminant(s) in a preparation may be assayed using any nucleic acid sequence as the template for polymerase amplification. The varying sensitivity of amplification between different templates indicates that the optimized oversulfated GAG sensitivity of the disclosed assay may be even greater than that illustrated in Example 1. Optimization of the assays provided herein for use in individual labs, with specific templates and enzyme preparations as well as specific lab procedures, is all well within the skill of one of ordinary skill in the art.

### Example 3

### Inhibition of DNA Dependent DNA Polymerases as a Method for Screening Heparin for Oversulfated Contaminant

This example describes a screen for oversulfated GAG contaminant in a preparation through use of any one of several DNA dependant DNA polymerases. The inhibition of DNA polymerases by heparin is well established (DiCioccio et al., Cancer Research, 38: 2401-2407, 1978, Yokota et al., J. Clin. Lab. Anal., 13: 133-140, 1999). Based on these similarities of action and the present disclosure, it is believed that oversulfated GAG will similarly inhibit heparin-sensitive DNA polymerases. In such instances, the presently disclosed resistance of such oversulfated GAGs to heparinase digestion will allow screening for such contaminants by the continued inhibition of such polymerases following heparinase treatment.

In particular examples, the inhibited DNA polymerases will be thermal stable polymerases. In such examples, any PCR-based nucleic acid amplification assay may be used to assay for polymerase activity. In such examples, polymerase activity can be detected by a fluorescent tag that is incorporated and detected spectroscopically as in quantitative real time PCR. Alternatively, polymerase activity can be detected by manual separation and quantification of PCR products by gel electrophoresis.

In other examples the particular DNA polymerase is not functional in PCR. The activity of such polymerases can be detected by any means possible to detect a specific in vitro replication product, such as by detection of an incorporated radioactive label. In such an example, following separation of replication products by electrophoresis, the incorporated radioactivity can be measured directly through radioactive emission counts, or indirectly through visualization and quantitation of replication products.

### Example 4

### Inhibition of DNA Dependent RNA Polymerase as a Method for Screening Heparin for Oversulfated Contaminant

This example describes a screen for oversulfated GAG contaminant in a preparation through use of any one of several DNA dependant RNA polymerases. The inhibition of RNA polymerases by heparin is well established (Pfeffer et al., J. Biol. Chem., 252:5403-5407, 1977). Based on these similarities of action and the present disclosure, it is likely that oversulfated GAG will similarly inhibit heparin-sensitive RNA polymerases. In such instances, the presently disclosed resistance of such oversulfated GAGs to heparin digestion will allow assay of the presence of such contaminants by the continued inhibition of such polymerases following heparinase treatment.

In a particular example, the activity of the E. coli RNA polymerase may be used to detect oversulfated GAG contaminant. In such an example, polymerase activity is measured through quantitation of produced RNA transcript by any method available to quantify RNA. In one example, RNA product can be directly measured through incorporation of a radioactive label into the transcript, followed by electrophoresis. RNA is then directly measured by the radioactive emission of a specified RNA product, or indirectly through visualization and quantitation. RNA product may also be measured indirectly by RT-PCR, either in conjunction with, or independent of, quantitative real time PCR. RNA product may also be measured indirectly by extension of a radioactively labeled primer by a reverse transcriptase. The resultant products would then be separated by electrophoresis, visualized, and quantified.

### Example 5

### Inhibition of Reverse Transcriptase as a Method for Screening Heparin for Oversulfated Contaminant

This example describes a screen for oversulfated GAG contaminant in a preparation through use of any one of several reverse transcriptases. The inhibition of reverse transcriptases by heparin is well established (Johnson et al., Biotechniques, 35: 1140-1144, 2003; Izraeli et al., Nucleic Acids Res., 19: 6051, 1991; Holodniy et al., J. Clin. Microbiol., 29: 676-679, 1991). Based on these similarities of action and the present disclosure, it is believed that oversulfated GAG will similarly inhibit heparin-sensitive reverse transcriptases. In such instances, the presently disclosed resistance of such oversulfated GAGs to heparin digestion will allow assay of the presence of such contaminants by the continued inhibition of such polymerases following heparinase treatment.

The activity of a reverse transcriptase is measured by various methods including extension of a radioactively labeled primer or as part of a RT-PCR reaction as described in the above examples.

### Example 6

### High Throughput Method for Screening Heparin Preparations for Oversulfated Contaminant

This example describes the use of the disclosed invention to assay for an oversulfated GAG contaminant by various high throughput methods. In particular examples the disclosed method is used to detect or quantify oversulfated GAG in a preparation by assaying for inhibition of a thermal stable DNA polymerase such as Taq polymerase. In this example, the method is used to screen through numerous samples.

Using an automated, semiautomated, or manual liquid handling device, samples from various heparin preparations may be dispensed into part or all of a microtiter plate. Using the same liquid handling methods, the method is performed as described. Concomitant use of a thermal cycler adapted to the particular microtiter plate format will allow for batch processing of heparinase treatment, nucleic acid amplification, and detection of (Taq) polymerase activity.

### Example 7

### Inhibition of a Nucleic Acid Polymerase as a Method for Screening a Therapeutic Preparation for Oversulfated Contaminant

This example describes the use of the disclosed methods to screen for oversulfated GAG contaminants in any therapeutic preparation.

Although the recent reports of severe adverse effects were associated with the administration of heparin, the study connecting the adverse effects to OS-CS noted the synthetic origin of the contaminant (Kishimoto et al., N. Engl. J Med., 358: 2457-2467, 2008). Therefore it is conceivable that other therapeutic agents could become contaminated with an oversulfated GAG. To detect such contamination, the disclosed method would be used as described, but the heparinase treatment step would be optional. Heparinase treatment optionally could be included to remove any trace amounts of heparin that might be in the (non-heparin) preparation to be screened. However, Applicants specifically envision embodiments where the assay would be based solely on the inhibition of polymerase activity by an oversulfated GAO contaminant and the comparison of polymerase activity between clean and potentially contaminated preparations.

Other than the heparinase treatment being optional, screening for oversulfated GAG contaminant in any given therapeutic sample would be accomplished using the inhibition of polymerase activity as described herein.

It will be apparent that the precise details of the methods, uses, and kits described may be varied or modified without departing from the spirit of the described invention. We claim all such modifications and variations that fall within the scope and spirit of the claims below.

1. A method of detecting and/or quantifying oversulfated glycosaminoglycan in a medical preparation lacking heparin, comprising determining whether the medical preparation lacking heparin reduces or inhibits activity of a nucleic acid polymerase.

2. A method of determining whether oversulfated glycosaminoglycan is present in a preparation containing heparin, comprising:
treating a sample of the preparation with sufficient heparinase to substantially degrade any heparin therein, thereby producing a heparinase-treated sample;
assaying activity of a nucleic acid polymerase in the presence of at least a portion of the heparinase-treated sample; and
comparing the activity of the nucleic acid polymerase in the presence of the heparinase-treated sample to the activity of the nucleic acid polymerase in the absence of the heparinase-treated sample, wherein a measurable reduction of the nucleic acid polymerase activity in the presence of heparinase-treated sample indicates that oversulfated glycosaminoglycan is present in the preparation.

3. A method of determining the relative quantity of an oversulfated glycosaminoglycan that is present in a preparation containing heparin, comprising:
treating a sample of the preparation with sufficient heparinase to substantially degrade any heparin therein, thereby producing a heparinase-treated sample;
assaying activity of a nucleic acid polymerase in the presence of increasing dilutions of the heparinase-treated sample; and
comparing the activity of the nucleic acid polymerase in the presence of the heparinase-treated sample to activity of the nucleic acid polymerase in the presence of known quantities of oversulfated glycosaminoglycan, wherein a similar reduction in the nucleic acid polymerase activity in the heparinase-treated sample and the nucleic acid polymerase activity in one of the known quantities of oversulfated glycosaminoglycan indicates the relative quantity of glycosaminoglycan in the preparation.

4. The method of claim 2, wherein the preparation is a therapeutic medical preparation.

5. The method of claim 1, wherein the medical preparation is a heparin preparation.

6. The method of claim 1, wherein the medical preparation is from a medical device

7. The method of claim 6, wherein the medical preparation is from a medical device containing or coated with heparin.

8. The method of claim 1, wherein the medical preparation is produced by removing heparin from a medical device containing or coated with heparin.

9. The method of claim 1, wherein the nucleic acid polymerase is a thermal stable DNA dependent DNA polymerase.

10. The method of claim 9, wherein the nucleic acid polymerase is Taq polymerase.

11. The method of claim 2, wherein assaying nucleic acid polymerase activity comprises running an in vitro nucleic acid amplification reaction.

12. The method of claim 11, wherein the in vitro nucleic acid amplification reaction comprises running a PCR amplification reaction, running a RT-PCR amplification reaction, or running a quantitative real time PCR amplification reaction.

13. The method of claim 2, wherein the nucleic acid polymerase activity in the presence of the heparinase-treated sample is reduced by a statistically significant amount of at least one standard deviation from the nucleic acid polymerase activity in the absence of the heparinase-treated sample.

14. The method of claim 2, wherein the nucleic acid polymerase activity in the presence of the heparinase-treated sample is reduced by a statistically significant amount of at least three standard deviations from the nucleic acid polymerase activity in the absence of the heparinase-treated sample.

15. The method of claim 1, wherein the oversulfated glycosaminoglycan comprises at least one oversulfated glycosaminoglycan of natural or synthetic origin.

16. The method of claim 1, wherein the oversulfated glycosaminoglycan comprises oversulfated chondroitin sulfate, oversulfated heparan sulfate, oversulfated dermatan sulfate, or two or more thereof.

17. The method of claim 2, wherein the heparinase comprises heparinase I, heparinase II, or a mixture thereof.

18. A kit for carrying out the method of claim 2, comprising: a container containing heparinase, nucleic acids, a nucleic acid polymerase, and instructions for comparing results of the method with a standard to provide a conclusion about the presence or quantity of oversulfated glycosaminoglycan contamination in a preparation.

19. The method of claim 1, wherein the medical preparation lacks heparin as a result of treating the medical preparation with heparinase.
